SEQUENCE LISTING (6 sequences)

SEQ ID NO: 1 - DNA, 20 bp, Artificial Sequence
  synthetic sequence
atgtggccatcttggatctc

SEQ ID NO: 2 - DNA, 20 bp, Artificial Sequence
  synthetic sequence
cacagccttgagcattgttg

SEQ ID NO: 3 - DNA, 20 bp, Artificial Sequence
  synthetic sequence
acacgacacgatgttttcca

SEQ ID NO: 4 - DNA, 20 bp, Artificial Sequence
  synthetic sequence
tcatggagcgtgctaacaac

SEQ ID NO: 5 - DNA, 21 bp, Artificial Sequence
  synthetic sequence
tccaactcgcagatccaagag

SEQ ID NO: 6 - DNA, 17 bp, Artificial Sequence
  synthetic sequence
cgacctgcgccctgttt

(19) United States
(12) Patent Application Publication
(10) Pub. No.: US 2014/0235484 A1
(43) Pub. Date: Aug. 21, 2014

(54) METHODS FOR DETECTION AND DIFFERENTIATION OF ORIGIN OF VIRAL DNA

(71) Applicant: THE JOHNS HOPKINS UNIVERSITY, Baltimore, MD (US)

(72) Inventors: Richard F. Ambinder, Lutherville, MD (US); Meir Shamay, Baltimore, MD (US)

(73) Assignee: THE JOHNS HOPKINS UNIVERSITY, Baltimore, MD (US)

(21) Appl. No.: 14/182,713

(22) Filed: Feb. 18, 2014

Related U.S. Application Data
(60) Provisional application No. 61/766,355, filed on Feb. 19, 2013.

(51) Int. Cl. C12Q 1/70; C12Q 1/68

(52) U.S. Cl. CPC C12Q 1/705; C12Q 1/70; C12Q 1/6886; C12Q 1/703
USPC 506/9; 435/5

(57) ABSTRACT

The invention disclosed herein provides methods for identifying the origin of viral DNA in a sample by detecting the methylation state of at least one CpG site on a target site of the genome of a virus of interest, and determining whether the origin of the viral DNA is from a cell, such as a tumor cell, or a virion, wherein when the methylation state is positive for methylation, the viral DNA is from a cell, and wherein when the methylation state is negative for methylation, the viral DNA is from a virion. Use of these methods for diagnosis or monitoring, and/or treatment of a subject infected with a virus known to cause cancer and related disease are also provided.

### REFERENCE TO RELATED APPLICATIONS

This application claims the benefit of U.S. Provisional Patent Application No. 61/766,355, filed on Feb. 19, 2013, and the content of the aforementioned application is herein incorporated by reference in its entirety.

### STATEMENT OF GOVERNMENTAL INTEREST

This invention was made with government support under grant nos. P50CA96888, U01CA121947, P30CA006973 and P01CA113239 awarded by the National Institutes of Health. The government has certain rights in the invention.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Feb. 6, 2014, is named P11863-03_ST25.txt and is 1,407 bytes in size.

### BACKGROUND OF THE INVENTION

Kaposi's sarcoma herpes virus (KSHV also known as HHV8) is associated with tumor cells in all forms of Kaposi's sarcoma (KS). KS is a tumor characterized by neovascular proliferation. It commonly presents as cutaneous lesions but lymphadenopathy, gut and lung involvement are not unusual. Physical exam and X-ray have been the major tools for assessing tumor. However, hyperpigmentation associated with cutaneous lesions persists for months or years after tumor response so that visual assessment is sometimes misleading. Edema, particularly in the legs, may result from tumor infiltration of the skin, obstruction of lymphatics associated with nodal involvement, or lymphatic scarring resulting from tumor. After chemotherapy, severe and sometimes disabling edema may persist. In some instances, evidence of tumor persistence would lead to further chemotherapy—but distinguishing residual lymphatic scarring from lymphatic obstruction associated with tumor in edematous legs is not easy.

Epstein-Barr virus is in the herpes family of viruses and most people will become infected with EBV sometimes during their lives. In the United States, as many as 95 percent of adults between 35 and 40 years of age have been infected. Infants become susceptible to EBV as soon as maternal protection present at birth disappears. EBV causes infectious mononucleosis and diseases such as Hodgkin's lymphoma, Burkitt's lymphoma, nasopharyngeal carcinoma, and conditions associated with human immunodeficiency virus (HIV) such as hairy leukoplakia and central nervous system lymphomas. There is evidence that infection with the virus is associated with a higher risk of certain autoimmune diseases, especially dermatomyositis, systemic lupus erythematosus, rheumatoid arthritis, Sjögren's syndrome, and multiple sclerosis.

Therefore there still exists a need for better tools for assessing the origin of viral DNA and its use in clinical applications such as the determination of tumor persistence or progression that are viral in origin, and that would be useful in guiding laboratory and clinical decision making.

### SUMMARY OF THE INVENTION

In accordance with an embodiment, the present invention provides a method for identifying the origin of viral DNA in a sample comprising: a) obtaining a biological sample comprising viral DNA, b) detecting the methylation state of at least one CpG site on a target site of the genome of the virus, and c) determining whether the origin of the viral DNA is from a cell or a virion, wherein when the methylation state of at least one CpG site on the target site is positive for methylation, the determination is made that the viral DNA is from a cell, and wherein when the methylation state of at least one CpG site on the target site is negative for methylation, the determination is made that the viral DNA is from a virion.

In accordance with another embodiment, the present invention provides a method for identifying the origin of viral DNA in a subject infected with a virus comprising: a) obtaining a biological sample comprising viral DNA from the subject, b) detecting the methylation state of at least one CpG site on a target site of the genome of the virus, and c) determining whether the origin of the viral DNA is from a cell or a virion, wherein when the methylation state of at least one CpG site on the target site is positive for methylation, the determination is made that the viral DNA is from a cell, and wherein when the methylation state of at least one CpG site on the target site is negative for methylation, the determination is made that the viral DNA is from a virion.

In accordance with yet another embodiment, the present invention provides a method of diagnosis of Kaposi's sarcoma in a subject infected with KSHV comprising: a) obtaining a biological sample comprising viral DNA from the subject, b) detecting the methylation state of at least one CpG site on a target site of the genome of the virus, and c) determining whether the origin of the viral DNA is from a cell or a virion, wherein when the methylation state of at least one CpG site on the target site is positive for methylation, the viral DNA is from a cell, and the patient is diagnosed as having Kaposi's sarcoma.

In accordance with a further embodiment, the present invention provides a method of diagnosis of primary effusion lymphoma in a subject infected with KSHV comprising: a) obtaining a biological sample comprising viral DNA from the subject, b) detecting the methylation state of at least one CpG site on a target site of the genome of the virus, and c) determining whether the origin of the viral DNA is from a cell or a virion, wherein when the methylation state is positive for methylation, the viral DNA is from a cell, and wherein when the methylation state is negative for methylation, the viral DNA is from a virion, and wherein when the methylation state is positive, the patient is diagnosed as having primary effusion lymphoma.

In accordance with a still further embodiment, the present invention provides a method of diagnosis of nasopharyngeal cancer in a subject infected with Epstein-Barr Virus comprising: a) obtaining a biological sample comprising viral DNA from the subject, b) detecting the methylation state of at least one CpG site on a target site of the genome of the virus, and c) determining whether the origin of the viral DNA is from a cell or a virion wherein when the methylation state of at least one CpG site on the target site is positive for methylation, the viral DNA is from a cell, and wherein when the methylation state is negative for methylation, the viral DNA is from a virion, and wherein when the methylation state is positive, the patient is diagnosed as having nasopharyngeal cancer.

### BRIEF DESCRIPTION OF THE DRAWINGS

FIG. 1 shows that paramagnetic beads linked to methylCpG binding domain protein 2 (MBD2-beads) distinguish between unmethylated virion DNA and methylated KSHV episomal DNA. DNA isolated from purified KSHV virions or from latently infected BC-3 cells were subjected to binding on the MBD2-beads. DNA isolated from the non-captured fraction (NC), washes (300 mM and 450 mM) and the elution (2000 mM) was subjected to real-time PCR with primers that amplify a region in the K8 ORF (1A), ORF 23 (1B), and ORF 64 (1C). Each column represents the amount of DNA in the indicated fraction relative to the total DNA detected (100%). Standard deviation of three independent real-time PCR reactions is indicated. (1D) A schematic representation of the KSHV genome. The nucleotide positions within the KSHV genome (Human herpesvirus 8 strain GK18, AF148805) of the amplified regions are indicated.

FIG. 2 depicts the analysis of the methylation status of KSHV DNA in patients with KS and primary effusion lymphoma (PEL) patients. DNA isolated from the blood from KS patients and from the blood or ascites fluid from PEL patients was subjected to binding on the methyl-CpG binding domain 2 (MBD2) beads. DNA isolated from the non-captured fraction (NC), washes (300 mM and 450 mM) and the elution (2000 mM) was subjected to real-time PCR with primers that amplify a region in ORF64.

FIG. 3 (A) DNA isolated from purified EBV virions or from latently infected Raji cells were subjected to binding to paramagnetic beads linked to methylCpG binding protein. DNA isolated from the non-captured fraction (NC), washes (300 mM and 450 mM) and the elution (2000 mM) was subjected to real-time PCR with primers that amplify a region in EBV BamW. (B) DNA isolated from the plasma of an AIDS patient and the plasma of a patient with EBV(+) Hodgkin lymphoma was subjected to binding to paramagnetic beads linked to methylCpG binding protein. DNA isolated from the different fractions was amplified as in 3A.

### DETAILED DESCRIPTION OF THE INVENTION

Tumors are recognized as a source of cell-free (cf) DNA in blood. Viral DNA can be released into blood from tumor and other cells as other cellular DNA or can be released packaged in virions, for example, from infected B cells.

In accordance with one or more embodiments, the present invention provides rapid and novel methods of detection of CpG methylated viral DNA in biological samples, including clinical samples from patients who are infected with, or are suspected of being infected with a virus known to cause a neoplasia or tumor in a mammal.

In accordance with an embodiment, the present invention provides a method for identifying the origin of viral DNA in a sample comprising a) obtaining a biological sample comprising viral DNA, b) detecting the methylation state of at least one CpG site on a target site of the genome of the virus, and c) determining whether the origin of the viral DNA is from a cell or a virion, wherein when the methylation state of at least one CpG site on the target site is positive for methylation, the viral DNA is from a cell, and wherein when the methylation state of at least one CpG site on the target site is negative for methylation, the viral DNA is from a virion.

It is understood that the methods disclosed in the present invention, that when it is determined that the origin of the viral DNA is from a cell, and the virus is associated with cancer, the cell can be a cancer cell. The type of cancer cell is not necessarily limited, and can include those types of cancers that are understood to be caused by viral infection. Examples include, but are not limited to Kaposi's sarcoma, cervical cancer, primary effusive lymphoma, Burkitt's lymphoma, nasopharyngeal cancer, T-cell lymphoma/leukemia hepatocellular carcinoma (HCC), adult T-cell leukemia, skin cancer in patients with epidermodysplasia verruciformis (EV), head and neck cancers, other anogenital cancers, post-transplant lymphomas, Hodgkin's disease, brain cancer, bone cancer, mesothelioma, prostate cancer, germ cell tumors, breast cancer, ovarian cancer, melanoma, gastrointestinal cancer, lung cancer, myeloma and others.

In accordance with other embodiments, there is at least one target site or gene from the viral DNA of interest that has at least one CpG site which is capable of methylation. In some embodiments, there can be two or more target sites or genes from the viral DNA of interest that has at least one CpG site which is capable of methylation. In some other embodiments, a target site or gene from the viral DNA which is not capable of methylation can be used as a control.

Methods of detecting the presence of cancer in a subject and methods of monitoring the treatment of cancer in a subject are further provided by the invention. In an embodiment, a method of monitoring the treatment of a subject undergoing cancer chemotherapy comprises a) obtaining a biological sample comprising viral DNA, b) detecting the methylation state of at least one CpG site on a target site of the genome of the virus, and c) determining whether the origin of the viral DNA is from a cell or a virion, wherein when the methylation state is positive for methylation, the viral DNA is from a cell, and wherein when the methylation state is negative for methylation, the viral DNA is from a virion, and when the methylation state is positive, the subject is diagnosed as needing further or continued cancer treatment or chemotherapy.

In accordance with an embodiment, the present invention provides a method of identifying the origin of viral DNA in a subject comprising: a) obtaining a biological sample from the subject; b) purifying DNA from the biological sample; c) allowing the DNA from b) to come in contact with a probe which is capable of specifically binding to a methylated CpG locus of the DNA in the sample and separating the methylated DNA from the unmethylated DNA; d) identifying whether the at least one target site is methylated from the DNA bound to the probe of c) and e) determining whether the origin of the viral DNA is from a cell when the methylation state of at least one CpG site on at least one target site of the genome of the virus is positive.

It will be understood that the term “biological sample” or “biological fluid” includes, but is not limited to, any quantity of a substance from a living or formerly living patient or mammal. Such substances include, but are not limited to, blood, serum, plasma, ascites, urine, cells, organs, tissues, bone, bone marrow, lymph, lymph nodes, synovial tissue, chondrocytes, synovial macrophages, endothelial cells, and skin.

In accordance with one or more embodiments, the methods disclosed herein are understood to include viral DNA from a species of virus that is associated with causing a neoplasia or tumor in a mammal. Such virus families include, but are not limited to Hepadnaviridae, Herpesviridae, and Papillomaviridae. Specific viruses include Epstein-Barr Virus (EBV), Kaposi's Sarcoma-Associated Herpes Virus (KSHV), Human T-Cell Leukemia Virus (HTLV-1), Hepatitis C Virus (HCV), Human Papillomavirus (HPV), and Hepatitis B Virus (HBV).

The term “target site” as used herein, means one or more regions of the viral genome that are analyzed for CpG methylation. In certain embodiments, the species of virus is KSHV and the target site of the virus DNA is selected from the group consisting of ORF64, ORF23 and K8. In accordance with an embodiment, at least one, two or all three target sites can be used to analyze the viral DNA. In another embodiment, where the virus of interest is EBV, the gene target is BamW. There is no upper limit to the number of target sites used in accordance with the methods of the invention.

“Probe” as used herein may mean an oligonucleotide capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. There may be any number of base pair mismatches which will interfere with hybridization between the target sequence and the single stranded nucleic acids described herein. However, if the number of mutations is so great that no hybridization can occur under even the least stringent of hybridization conditions, the sequence is not a complementary target sequence. A probe may be single stranded or partially single and partially double stranded. The strandedness of the probe is dictated by the structure, composition, and properties of the target sequence. Probes may be directly labeled or indirectly labeled such as with biotin to which a streptavidin complex may later bind. In accordance with one or more embodiments, the term “probe” also means an oligonucleotide which is capable of specifically binding to a CpG locus which can be methylated. The DNA gene target or probes of the present invention are used to determine the methylation status of at least one CpG dinucleotide sequence of at least one target gene as described herein.

It will be understood by those of ordinary skill, that there are a number of ways to detect DNA methylation, and these are known in the art. Examples of preferred methods of detection of methylation of DNA in a sample include the use of QMSP, oligonucleotide methylation tiling arrays, paramagnetic beads linked to MBD2, i.e., BeadChip assays and HPLC/MS methods. Other methods include methylation-specific multiplex ligation-dependent probe amplification (MS-MPLA), bisulfate sequencing, and assays using antibodies to DNA methylation, i.e., ELISA assays. The methylation state information gathered from these methods can be generated using any type of microprocessor or computing device.

Examples of preferred detection methods include detection of CpG methylation of the viral DNA in the sample by the use of paramagnetic beads linked to MBD2.

In an embodiment, the DNA of the sample, after extraction and purification, subjected to a slurry or MBD2 beads and the non-captured fraction is eluted and collected, followed by elution and collection of fractions eluted with increasing concentrations of NaCl solutions (e.g. 300 mM, 450 mM and 2000 mM). The eluted fractions are then concentrated and subjected to RT-PCR using probes specific for the viral target genes of interest. The fractions are then compared. DNA in the non-captured fraction is not methylated, whereas DNA in any of the NaCl elution fractions is considered methylated.

As used herein, the term “methylation state” means the detection of one or more methyl groups in a CpG site in a target site of the viral DNA.

By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

In an embodiment, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids used as primers in embodiments of the present invention can be constructed based on chemical synthesis and/or enzymatic ligation reactions using procedures known in the art. See, for example, Sambrook et al. (eds.), Molecular Cloning, A Laboratory Manual, 3^{rd} Edition, Cold Spring Harbor Laboratory Press, New York (2001) and Ausubel et al., Current Protocols in Molecular Biology, Greene Publishing Associates and John Wiley & Sons, NY (1994). For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

The nucleotide sequences used herein are those which hybridize under stringent conditions preferably hybridizes under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C.

As used herein, the term “host cell” refers to any type of cell that can contain the viral DNA disclosed herein. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, BC-3 cells, and the like. In an embodiment, the host cell is preferably a mammalian cell. Most preferably, the host cell is a human cell or human cell line. The host cell can be of any cell type, can originate from any type of tissue, and can be of any developmental stage.

The term “isolated and purified” as used herein means a protein that is essentially free of association with other proteins or polypeptides, e.g., as a naturally occurring protein that has been separated from cellular and other contaminants by the use of antibodies or other methods or as a purification product of a recombinant host cell culture.

The term “biologically active” as used herein means an enzyme or protein having structural, regulatory, or biochemical functions of a naturally occurring molecule.

The term “subject” used herein includes animals such as humans, sheep, horses, cattle, pigs, monkeys, dogs, cats, rats, mice and other mammals.

The term “reacting” in the context of the embodiments of the present invention means placing compounds or reactants in proximity to each other, such as in solution, in order for a chemical reaction to occur between the reactants.

The term “virion,” as used herein is interchangeable with the term “virus” or “viral particle.”

In accordance with another embodiment of the present invention, it will be understood that the term “biological sample” or “biological fluid” includes, but is not limited to, any quantity of a substance from a living or formerly living patient or mammal. Such substances include, but are not limited to, blood, serum, ascites fluid, plasma, urine, cells, organs, tissues, bone, bone marrow, lymph, lymph nodes, synovial tissue, chondrocytes, synovial macrophages, endothelial cells, and skin.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

A probe is also provided comprising a nucleic acid described herein. Probes may be used for screening and diagnostic methods, as outlined below. The probes may be attached or immobilized to a solid substrate or apparatus, such as a biochip.

The probe may have a length of from 8 to 500, 10 to 100 or 20 to 60 nucleotides. The probe may also have a length of at least 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 220, 240, 260, 280 or 300 nucleotides. The probe may further comprise a linker sequence of from 10-60 nucleotides.

In accordance with one or more embodiments, the arrays of the present invention further comprise at least one randomly-generated oligonucleotide probe sequence used as a negative control; at least one oligonucleotide sequence derived from a housekeeping gene, used as a negative control for total DNA degradation; at least one randomly-generated sequence used as a positive control; and a series of dilutions of at least one positive control sequence used as saturation controls; wherein at least one positive control sequence is positioned on the array to indicate orientation of the array.

A biochip is also provided. The biochip is an apparatus which, in certain embodiments, comprises a solid substrate comprising an attached probe or plurality of probes described herein. The probes may be capable of hybridizing to a target sequence under stringent hybridization conditions. The probes may be attached at spatially defined address on the substrate. More than one probe per target sequence may be used, with either overlapping probes or probes to different sections of a particular target sequence. In an embodiment, two or more probes per target sequence are used. The probes may be capable of hybridizing to target sequences associated with a single disorder.

The probes may be attached to the biochip in a wide variety of ways, as will be appreciated by those in the art. The probes may either be synthesized first, with subsequent attachment to the biochip, or may be directly synthesized on the biochip.

In accordance with one or more embodiments, the biochips of the present invention are capable of hybridizing to a target sequence under stringent hybridization conditions and attached at spatially defined address on the substrate.

In accordance with some embodiments the beads, arrays or chips used to identify methylated target sites are probes capable of binding a nucleotide sequence or portion or fragment thereof of the viral gene of interest. In an embodiment, where the virus of interest is KSHV, the gene targets are K8, ORF23 and ORF 64. In another embodiment, where the virus of interest is EBV, the gene target is BamW.

The solid substrate may be a material that may be modified to contain discrete individual sites appropriate for the attachment or association of the probes and is amenable to at least one detection method. Representative examples of substrates include glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses and plastics. The substrates may allow optical detection without appreciably fluorescing.

The substrate may be planar, although other configurations of substrates may be used as well. For example, probes may be placed on the inside surface of a tube, for flow-through sample analysis to minimize sample volume. Similarly, the substrate may be flexible, such as a flexible foam, including closed cell foams made of particular plastics.

The biochip and the probe may be derivatized with chemical functional groups for subsequent attachment of the two. For example, the biochip may be derivatized with a chemical functional group including, but not limited to, amino groups, carboxyl groups, oxo groups or thiol groups. Using these functional groups, the probes may be attached using functional groups on the probes either directly or indirectly using linkers. The probes may be attached to the solid support by either the 5′ terminus, 3′ terminus, or via an internal nucleotide.

The probe may also be attached to the solid support non-covalently. For example, biotinylated oligonucleotides can be made, which may bind to surfaces covalently coated with streptavidin, resulting in attachment. Alternatively, probes may be synthesized on the surface using techniques such as photopolymerization and photolithography.

Exemplary biochips of the present invention include an organized assortment of oligonucleotide probes described above immobilized onto an appropriate platform. In accordance with another embodiment, the biochip of the present invention can also include one or more positive or negative controls. For example, oligonucleotides with randomized sequences can be used as positive controls, indicating orientation of the biochip based on where they are placed on the biochip, and providing controls for the detection time of the biochip when it is used for detecting methylated gene targets from a sample.

Embodiments of the biochip can be made in the following manner. The oligonucleotide probes to be included in the biochip are selected and obtained. The probes can be selected, for example, based on a particular subset target DNA genes of interest. The probes can be synthesized using methods and materials known to those skilled in the art, or they can be synthesized by and obtained from a commercial source, such as GeneScript USA (Piscataway, N.J.).

Each discrete probe is then attached to an appropriate platform in a discrete location, to provide an organized array of probes. Appropriate platforms include membranes and glass slides. Appropriate membranes include, for example, nylon membranes and nitrocellulose membranes. The probes are attached to the platform using methods and materials known to those skilled in the art. Briefly, the probes can be attached to the platform by synthesizing the probes directly on the platform, or probe-spotting using a contact or non-contact printing system. Probe-spotting can be accomplished using any of several commercially available systems, such as the GeneMachines™ OmniGrid (San Carlos, Calif.).

The biochips are scanned, for example, using an Epson Expression 1680 Scanner (Seiko Epson Corporation, Long Beach, Calif.) at a resolution of about 1500 dpi and 16-bit grayscale. The biochip images can be analyzed using Array-Pro Analyzer (Media Cybernetics, Inc., Silver Spring, Md.) software. Because the identity of the target DNA gene probes on the biochip are known, the sample can be identified as including particular target DNA genes when spots of hybridized target DNA genes-and-probes are visualized. Additionally, the density of the spots can be obtained and used to quantitate the identified target DNA genes in the sample.

Methods of diagnosis are also provided. The methods comprise detecting a methylation state of one or more target genes discussed above in a biological sample. Diagnosis of a disease state in a subject may allow for prognosis and selection of therapeutic strategy. In an embodiment, the methods of the present invention can be used to determine if a subject infected with a virus has developed a cancer caused by the virus. In that situation, the determination of methylated viral DNA in the sample of the subject would indicate the likelihood of a tumor cell being the source of the methylated DNA and the patient would be diagnosed as having that cancer or tumor and could initiate treatment, such as chemotherapy.

In an alternative embodiment, the inventive methods can be used to monitor progression of a viral derived cancer by analyzing the amount of methylated viral DNA in the sample. In such a method, an increase in the relative amount of methylated viral DNA in the sample could indicate tumor progression, and a decrease in the relative amount of methylated viral DNA in the sample could indicate tumor remission or eventually disappearance. This analysis can be performed before, during or after chemotherapy and/or surgery and/or radiation treatment in the subject. The methods can also be used to determine whether chemotherapeutic dosage changes need to be made relative to the increase or decrease the relative amount of methylated viral DNA in the sample from the subject. The decreasing relative amount of methylated viral DNA in response to chemotherapy can indicate treatment is effective and the opposite would indicate the treatment was not effective and the dosage and/or type of therapy may need to be altered.

A kit is also provided comprising an array of oligonucleotides as described herein, or portions or fragments thereof, as well as a biochip as described herein, along with any or all of the following: assay reagents, buffers, probes and/or primers, and sterile saline or another pharmaceutically acceptable emulsion and suspension base. In addition, the kits may include instructional materials containing directions (e.g., protocols) for the practice of the methods described herein.

### EXAMPLES

Cell culture, control DNA samples, and DNA isolation. BC-3 is a primary effusion lymphoma cell line that harbors KSHV episomes. Purified virions were prepared from the supernatant of BC-3 cultures induced with sodium butyrate 0.3 ng/ml (for the initial 24 hours) and 12-O-fetradecanoylphorbol-13-acetate (TPA) 20 ng/ml for 5 days. After five days the cell suspension was transferred into 50 ml conical tubes and centrifuged (3500 RPM for 20 minutes at 4° C.). Clarified media were centrifuged at 15000 RPM for 35 minutes at 4° C. DNA was extracted from virus pellets according to manufacturer protocol (QIAampDNA Blood Mini Kit, QIAGEN).

Specimens. Pre-treatment plasma specimens from patients with AIDS KS enrolled on AIDS Malignancy Consortium trial 036 were studied as well as plasma and ascites specimens from patients with AIDS PEL. For the EBV testing, specimens, plasma specimens were obtained from AIDS patients without lymphoma and patients having EBV-associated Hodgkin lymphoma. Specimens were obtained with written informed consent and with approval from the relevant institutional review boards.

Methylated DNA Enrichment. Extracted DNA was added to 10 μl of MBD-Bead slurry (MethylMiner DNA Enrichment Kit, Invitrogen, Carlsbad, Calif.) and incubated on a rotating mixer for 1 hour. The DNA in the non-captured fraction (NC), washes (300 mM and 450 mM NaCl) and the elution (2000 mM NaCl) was ethanol precipitated, resuspended in water, and subjected to real-time PCR with Power SYBR Green PCR master mix (Applied Biosystems) and primers for KSHV ORF 64 (sense: ATGTGGCCATCTTGGATCTC (SEQ ID NO: 1) antisense: CACAGCCTTGAGCATTGTTG (SEQ ID NO: 2)), ORF23 (sense: ACACGACACGATGTTTTCCA (SEQ ID NO: 3), antisense: TCATGGAGCGTGCTAACAAC (SEQ ID NO: 4)), and K8 (sense: TCCAACTCGCAGATCCAAGAG (SEQ ID NO: 5), antisense: CGACCTGCGCCCTGTTT (SEQ ID NO: 6)). KSHV copy numbers were measured by using real-time PCR with primers and a probe that targeted the K8 region, as described previously in J. Clin. Oncol., 27:2496-502 (2009).

For EBV testing, the DNA purified as above was also subjected to real-time PCR as above using primers specific for the BamW gene.

### Example 1

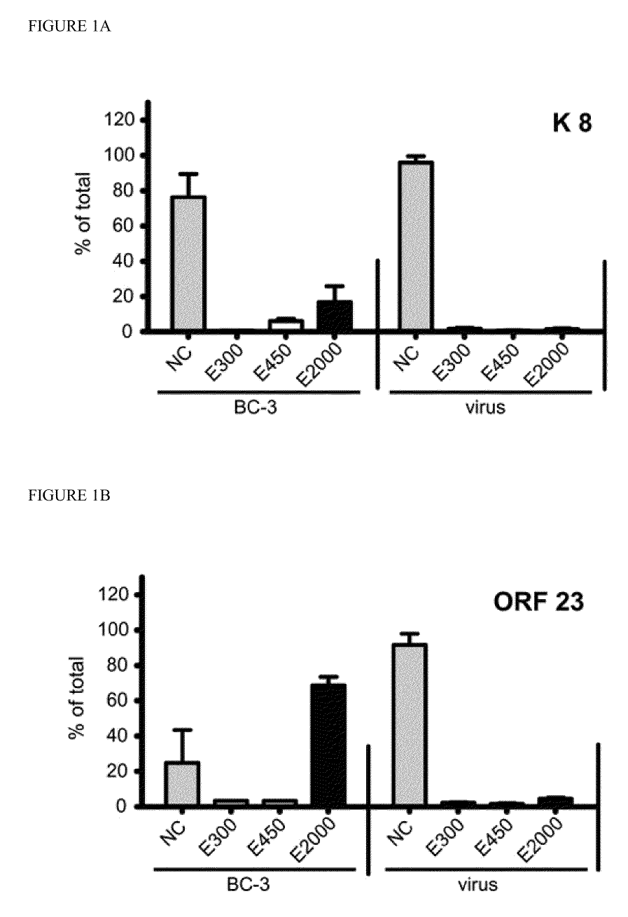

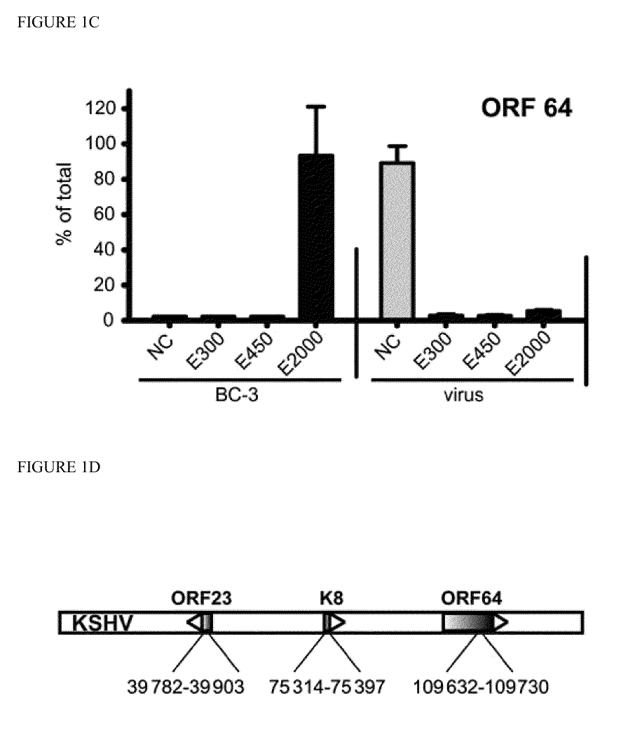

Paramagnetic beads coupled to the methyl-CpG binding domain of MBD2 were applied to KSHV virion and KSHV cell-derived DNA using the methods of the present invention. Non-capture (NC), wash (E15, E400) and high salt eluate (E2000) fractions were evaluated by real time PCR with three sets of KSHV primers (FIG. 1). Virion DNA was never captured by the resin regardless of the region of the viral genome targeted by primers for amplification consistent with the expectation that virion DNA was not methylated. Cellular viral DNA showed major differences as a function of the region of the viral genome analyzed. DNA from the K8 region was never captured by the resin. The DNA from the ORF23 and ORF64 regions were captured, however, only some of the DNA from the ORF23 region was captured, while all of the DNA from the ORF64 region was captured. Thus, MBD2 paramagnetic beads in combination with appropriate PCR primers of the present invention can be used to distinguish viral sequences derived from virion versus viral sequences from cellular DNA.

The sensitivity of the methods of the present invention were assessed in reconstruction experiments by mixing virion and BC-3 DNA. BC-3 DNA could consistently be distinguished from virion DNA when it constituted 5% or more of the total viral DNA in the sample (data not shown).

### Example 2

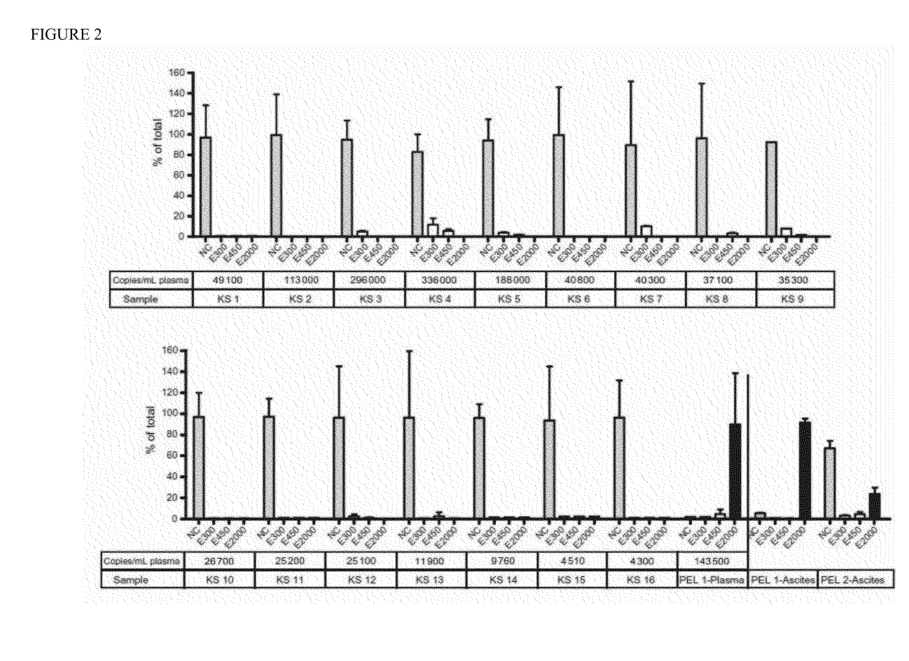

The methods of the present invention were then applied to DNA isolated from plasma from 16 patients with AIDS KS, plasma from a patient with AIDS primary effusion lymphoma (PEL); and DNA extracted from malignant ascites from two patients with PEL. As seen in FIG. 2, using the MBD2 beads, no CpG methylation was detected in plasma of any KS patient, but CpG methylation was detected in DNA from the plasma of a patient with PEL (FIG. 2). CpG methylated DNA was also detected in the ascites from both PEL patients.

In cell-free DNA samples (cfDNA) from blood of AIDS KS patients, the only KSHV DNA detected failed to bind to the MBD2-beads, which is consistent with absence of CpG methylation. These results indicate that the viral DNA sequences detected in plasma by the methods of the present invention are virion DNA. In contrast, in blood from a patient with PEL and in ascites from two patients with PEL, the methods of the present invention detected CpG methylation of viral sequences. Thus, KSHV derived from tumor cells can be detected in cfDNA in the blood of a patient with PEL. Moreover, tumor cell DNA is not readily detected by the present invention in the cfDNA of patients with AIDS KS in the absence of concurrent lymphoma.

The methods of the present invention were successfully used to show that the cfDNA that predominates in AIDS KS patients in the United States, without visceral KS lesions, is almost exclusively of virion origin rather than of tumor origin.

### Example 3

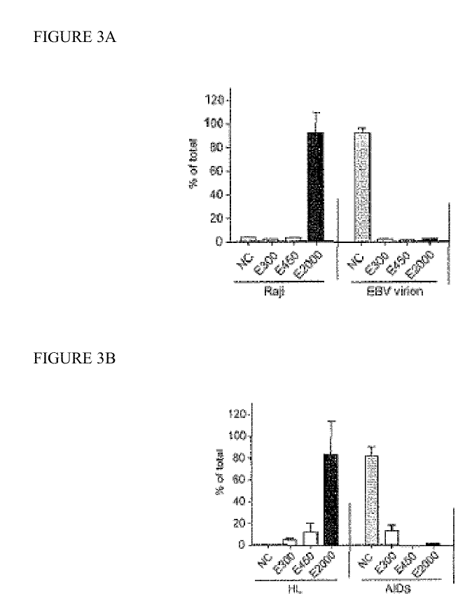

MBD2 paramagnetic beads linked to methylCpG binding protein were used to separate virion and cell-derived viral DNA. DNA isolated from EBV (FIG. 3A) virions failed to bind to the methylCpG binding protein and were detected only in the non-captured (NC) fractions, while DNA isolated from latently infected cell lines were detected predominantly in the bound fractions (E2000, high salt elute). Unmethylated EBV DNA, presumably virion DNA, was detected in the plasma of 3 AIDS patients without lymphoma, while methylated DNA was detected in the blood of 3 patients with EBV-associated Hodgkin lymphoma (HL) (without HIV infection) (FIG. 3B).

The present invention shows that tumor derived viral DNA can be distinguished from virion associated viral DNA based on preferential binding to methylCpG binding protein. Tumor derived viral DNA was predominantly present in the blood from patients with Hodgkin-Lymphoma, but not in patients without EBV associated malignancy. This technique may be applied to detect tumor derived viral DNA in the blood of patients with EBV associated malignancies.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for identifying the origin of viral DNA in a sample comprising:
a) obtaining a biological sample comprising viral DNA;
b) purifying the DNA from a);
c) allowing the DNA from b) to contact at least one or more paramagnetic beads bound with methylCpG binding protein;
d) detecting the methylation state of at least one CpG site on a target site of the genome of the virus; and
e) determining whether the origin of the viral DNA is from a cell or a virion, wherein when the methylation state of at least one CpG site on the target site is positive for methylation, the viral DNA is from a cell, and wherein when the methylation state of at least one CpG site on the target site is negative for methylation, the viral DNA is from a virion.

2. A method for identifying the origin of viral DNA in a subject infected with a virus comprising:
a) obtaining a biological sample comprising viral DNA;
b) purifying the DNA from a);
c) allowing the DNA from b) to contact at least one or more paramagnetic beads bound with methylCpG binding protein;
d) detecting the methylation state of at least one CpG site on a target site of the genome of the virus; and
e) determining whether the origin of the viral DNA is from a cell or a virion, wherein when the methylation state of at least one CpG site on the target site is positive for methylation, the viral DNA is from a cell, and wherein when the methylation state of at least one CpG site on the target site is negative for methylation, the viral DNA is from a virion.

3. The method of claim 2, wherein when it is determined that the origin of the viral DNA is from a cell, and the virus is associated with cancer, the cell is a cancer cell.

4. The method of claim 3, wherein the biological sample is selected from the group consisting of blood, serum, sputum, plasma, ascites, urine, cells, organs, tissues, bone, bone marrow, lymph, lymph nodes, synovial tissue, chondrocytes, synovial macrophages, endothelial cells, and skin.

5. The method of claim 4, wherein the viral DNA is from a species of virus that is associated with causing a neoplasia or tumor in a mammal.

6. The method of claim 5, wherein the species of virus is selected from the group consisting of Epstein-Barr Virus (EBV), Kaposi's Sarcoma-Associated Herpes Virus (KSHV), Human T-Cell Leukemia Virus (HTLV-1), Hepatitis C Virus (HCV), Human Papillomavirus (HPV), and Hepatitis B Virus (HBV).

7. The method of claim 6, wherein the species of virus is KSHV and the target site of the virus DNA is selected from the group consisting of ORF64, ORF23 and K8.

8. (canceled)

9. A method of diagnosis of Kaposi's sarcoma in a subject infected with KSHV comprising:
a) obtaining a biological sample comprising viral DNA from the subject;
b) purifying the DNA from a);
c) allowing the DNA from b) to contact at least one or more paramagnetic beads bound with methylCpG binding protein;
d)) determining whether the origin of the viral DNA is from a cell or a virion wherein when the methylation state of at least one CpG site on the target site is positive for methylation, the viral DNA is from a cell, and wherein when the methylation state of at least one CpG site on the target site is negative for methylation, the viral DNA is from a virion,
wherein when the methylation state is positive, the patient is diagnosed as having Kaposi's sarcoma.

10. A method of diagnosis of primary effusion lymphoma in a subject infected with KSHV comprising:
a) obtaining a biological sample comprising viral DNA from the subject;
b) purifying the DNA from a);
c) allowing the DNA from b) to contact at least one or more paramagnetic beads bound with methylCpG binding protein;
d)) determining whether the origin of the viral DNA is from a cell or a virion wherein when the methylation state of at least one CpG site on the target site is positive for methylation, the viral DNA is from a cell, and wherein when the methylation state of at least one CpG site on the target site is negative for methylation, the viral DNA is from a virion,
wherein when the methylation state of at least one CpG site on the target site is positive, the patient is diagnosed as having primary effusion lymphoma.

11. A method of diagnosis of nasopharyngeal cancer in a subject infected with Epstein-Barr Virus comprising:
a) obtaining a biological sample comprising viral DNA from the subject;
b) purifying the DNA from a);
c) allowing the DNA from b) to contact at least one or more paramagnetic beads bound with methylCpG binding protein;
d) determining whether the origin of the viral DNA is from a cell or a virion wherein when the methylation state is positive for methylation, the viral DNA is from a cell, and wherein when the methylation state is negative for methylation, the viral DNA is from a virion,
wherein when the methylation state of at least one CpG site on the target site is positive, the patient is diagnosed as having nasopharyngeal cancer.

12. The method of claim 9, wherein the target site is selected from the group of genes consisting of K8, ORF 23 and ORF 64.

13. The method of claim 10, wherein the target site is selected from the group of genes consisting of K8, ORF 23 and ORF 64.

14. The method of claim 11, wherein the target site is BamW.
